(19) United States
(12) Patent Application Publication
(10) Pub. No.: US 2019/0030290 A1
(43) Pub. Date: Jan. 31, 2019

(54) Vascular Access Needle for Guidewire Insertion

(71) Applicant: Aaron Ginster, Fox Point, WI (US)

(72) Inventor: Aaron Ginster, Fox Point, WI (US)

(21) Appl. No.: 16/033,056

(22) Filed: Jul. 11, 2018

Related U.S. Application Data
(60) Provisional application No. 62/538,321, filed on Jul. 28, 2017.

(51) Int. Cl. A61M 25/06

(52) U.S. Cl. CPC A61M 25/065; A61B 17/3417

(57) ABSTRACT

A vascular access needle for guidewire insertion. The needle comprises a barrel defined by a cylindrical wall. The barrel is hollow, defining a channel that extends along a central longitudinal axis of the needle. There is a bevel at the distal end with a lancet tip. The channel of the needle is configured so that it takes an oblique path at the distal end of the channel. As such, a guidewire inserted through the needle would exit the needle at an oblique angle. This configuration allows for improved vascular cannulation, especially in situations where the blood vessel is difficult to access.

### RELATED APPLICATIONS

This application claims the benefit of U.S. Provisional Patent Application No. 62/538,321 filed on 28 Jul. 2017, the contents of which are incorporated herein by reference.

### TECHNICAL FIELD

This invention relates to hollow bore needles used in medical practice.

### BACKGROUND

A central venous catheter (also known as a central line) placed into a large vein is commonly used for administering medication or fluids. Central venous catheters are useful in various situations such as when peripheral venous access is impossible, large volume fluid resuscitation or vasopressors is needed, or long-term intravenous antibiotics or chemotherapy is being administered. The central line catheter is inserted via an access route created by a hollow bore needle in conjunction with a guidewire. FIG. 1A shows (side view) an example of a conventional stainless steel hollow bore needle 10 that can be used for introducing a central line catheter. The needle 10 has a shaft portion 12 and at its distal end, there is a beveled tip 14. At the proximal end, there is a hub 16 having a Luer-type collar 18 for connecting with a Luer-type connection fitting.

FIG. 1B shows a close-up view (see-through) of the distal portion of the needle 10. As seen here, there is a hollow channel 24 created by the wall 22 of the needle 10. At the bevel 14 of the needle shaft 12, there is a lancet tip 20 that provides a sharp edge for cutting into skin and body tissue. FIG. 1C is a perspective view of the distal portion of the needle 10, showing more clearly the hollow channel 24 created by the wall 22 of the needle 10.

FIGS. 2A and 2B show how the needle 10 is used for providing the initial access path into the vein 35. In FIG. 2A, the needle 10 is punctured through the skin 30 at approximately 35° angle, advancing through the superficial fascia 31, through the fatty layer 32, and into the vein 35. To secure this access path into the vein 35, a blunt guidewire 28 is inserted through the needle hub 16 and passed through the hollow needle shaft 12 until it exits from the needle tip and into the lumen 34 of the vein 35. With the guidewire 28 held within the vein 35, the needle 10 is withdrawn backwards over the guidewire 28, leaving only the guidewire 28 in place. A central venous catheter can then be introduced into vein 35 over this guidewire 28.

However, this deep vein catheterization procedure can be complicated and often difficult to perform successfully. The source of much of the difficulty is getting the guidewire to thread parallel into the blood vessel. This is especially challenging in obese patients having thicker skin. This problem is illustrated in FIG. 2B. As seen here, for an obese patient, the fatty layer 32 is substantially thicker. This lengthens the distance that the needle 10 must traverse to reach the vein 35. Because of the resulting geometry, the needle 10 must be directed at a sharper angle for better aim and to reach the vein 35. With the guidewire 28 exiting the needle 10 on a straight line path, this sharper entry angle makes it more difficult for the guidewire 28 to cannulate into the lumen 34 of the vein 35.

Cannulation of the vein with a guidewire is needed to place a central line. Failure to successfully place the central line can result in delayed administration of lifesaving medications. In addition, efforts to insert the central line can result in accidental trauma, such as repeated perforations of the blood vessel, pneumothorax, perforated trachea, damage to other nearby blood vessels, and other inadvertent damage.

### SUMMARY

My invention provides a vascular access needle for guidewire insertion. This improved needle could result in a variety of benefits, including faster procedure time, fewer procedure attempts, earlier delivery of intravenous medications, or reduced incidence of complications. In one aspect, my invention is a hollow bore needle comprising a barrel defined by a cylindrical wall. The barrel encloses an internal channel that extends along a central longitudinal axis of the barrel. The barrel may have only a single internal channel. At the distal end of the barrel, there is a bevel portion of the barrel that comprises a lancet tip.

The shaft portion of the barrel is defined as the portion of the barrel that extends proximally from the bevel portion. Within the needle barrel, there is an internal lip that forms a ramp having an incline relative to the cylindrical wall. The path of the internal channel at the shaft portion of the barrel is parallel to the central longitudinal axis and at the internal lip, oblique relative to the central longitudinal axis. In some cases, the internal lip is angled in the range of 5-20° as it extends away from the cylindrical wall relative to the cylindrical wall or the central longitudinal axis; and in some cases, in the range of 7-15°. In some cases, the internal lip is located within 2.5 cm of the distal end of the barrel. The internal lip may form a partial obstruction within the needle barrel.

The needle barrel may be configured such that the outer surface of the barrel is parallel and coaxial to the central longitudinal axis along the entire length of the barrel, including the bevel portion. In some cases, the barrel has a diameter in the range of 18-24 gauge. The barrel of the needle may have any suitable length. For example, the length of the barrel (from hub to distal tip) could range from 2.5-12 cm. The barrel can be made of any suitable material. For example, the barrel could be made of a material comprising stainless steel.

In some cases, the hollow bore needle further comprises a Luer-type hub at the proximal end (i.e. the hub is configured to connect with a Luer-type connection fitting). The bevel portion of the needle barrel can have any suitable design. For example, the bevel portion can be angled in the range of 8-25° relative to the central longitudinal axis of the barrel.

In one embodiment of the hollow bore needle, the internal lip is located within the needle barrel at the bevel portion. The internal lip forms a inclined ramp that leads out to an opening at the bevel portion of the needle barrel.

In another embodiment of the hollow bore needle, there is a side opening in the cylindrical wall on the shaft portion of the needle that opens into the channel. In some cases, the side opening is located within a distance of 2.5 cm from the distal end of the barrel. The internal lip is located within the needle barrel at a distance within 2.5 cm from the distal end of the barrel. The internal lip forms an inclined ramp that leads out to the side opening on the needle barrel.

In another aspect, my invention is a vascular access kit comprising a hollow needle of my invention and a guidewire configured to travel through the channel of the hollow needle. The guidewire may have any suitable diameter, such as a diameter in the range of 0.014 inches (0.36 mm)-0.050 inches (1.3 mm). The kit may further include other conventional components, such as introducer sheaths, catheters, syringes, dilators, etc.

In another aspect, my invention is a method of providing an access path into a blood vessel using a hollow needle of my invention. The needle is applied to the patient's skin and punctured into the skin. In some embodiments, the needle may be inserted into the skin at an angle of greater than 45° relative to the skin surface or the blood vessel; and in some cases, at an angle greater than 60°.

The needle is advanced until the bevel portion or side opening of the barrel enters the lumen of the blood vessel. The method further comprises inserting a guidewire into the channel of the barrel at the proximal end of the needle and advancing the guidewire through the channel until it exits the barrel and into the lumen of the blood vessel. The guidewire exits the needle barrel, at an opening at the bevel portion or a side opening on the shaft portion, at an oblique angle relative to the central longitudinal axis of the needle barrel; in some cases, at an angle in the range of 20-90°; in some cases, in the range of 25-75°; and in some cases, in the range of 25-60°. The needle is then withdrawn over the guidewire, leaving the distal end of the guidewire in place within the lumen of the blood vessel.

This technique could be particularly useful in patients having a body mass index (BMI) in the overweight range (25-30) or in the obese range (greater than 30). This technique could be used in any type of blood vessel, including veins and arteries. Examples of central veins that could be targeted by this technique include internal jugular vein, subclavian vein, femoral vein, or other deeper vein. Examples of arteries that could be targeted include femoral artery or radial artery. Examples of types of catheters that could be inserted using this technique include triple lumen central lines, Cordis-type catheters, peripherally inserted central catheters (PICC), Hickman-type catheters, Groshong-type catheters, Quinton-type catheters, and implanted port catheters.

### BRIEF DESCRIPTION OF THE DRAWINGS

FIGS. 1A-C show a conventional hollow bore needle of the prior art. FIG. 1A shows a side view of the needle. FIG. 1B shows a close-up, see-through view of the distal portion of the needle. FIG. 1C shows a perspective view of the distal portion of the needle.

FIGS. 2A and 2B show how the needle of FIG. 1 is used for providing the initial access path into a vein.

FIGS. 3A-3C show an example needle of my invention. FIG. 3A shows a side view of the needle. FIG. 3B shows a close-up, see-through view of the distal portion of the needle. FIG. 3C shows a close-up, perspective view of the distal portion of the needle.

FIGS. 4A and 4B show how the needle of FIGS. 3A-3C can be used in conjunction with a guidewire to cannulate a vein. FIG. 4A shows a close-up, see-through view of the distal portion of the needle with a guidewire extending therethrough. FIG. 4B shows how the needle could be used for providing the initial access path into a vein.

FIG. 5 shows a particular example of how an internal lip could be formed.

FIGS. 6A-6C show another example needle of my invention. FIG. 6A shows a close-up, see-through view of the distal portion of the needle. FIGS. 6B and 6C show close-up, perspective views of the distal portion of the needle.

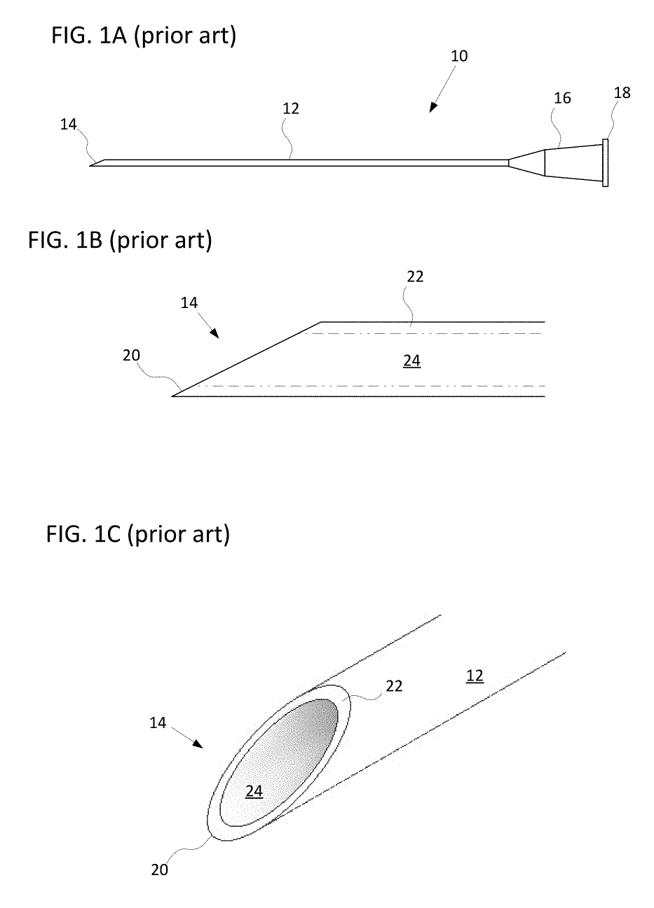

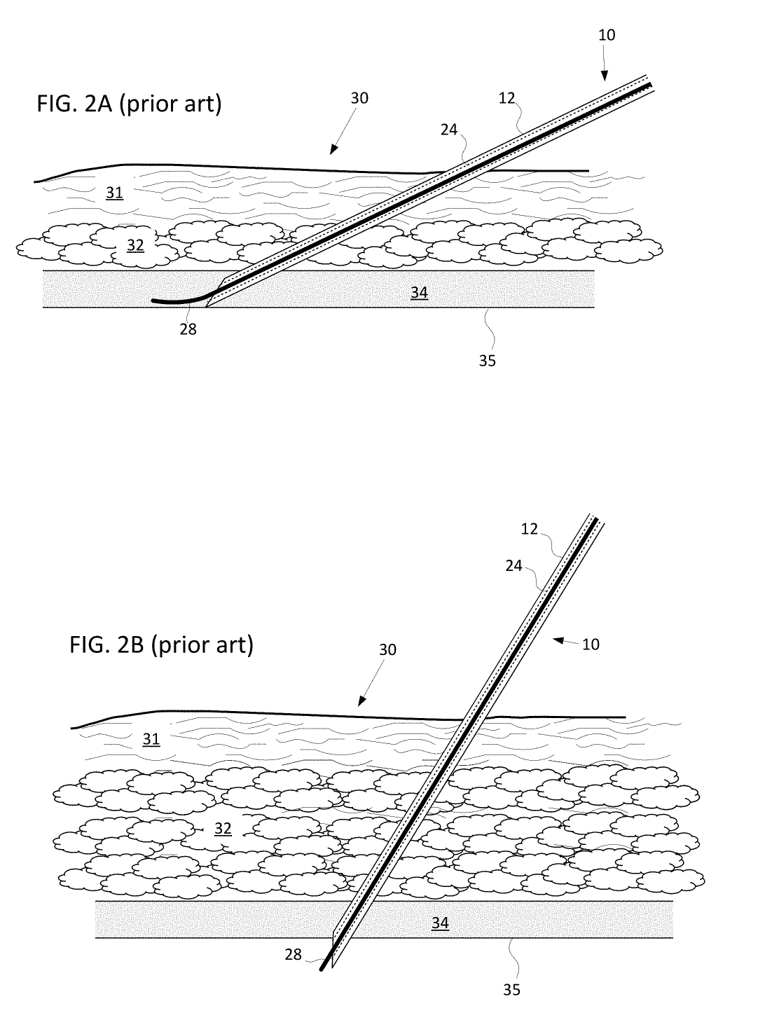

### DETAILED DESCRIPTION

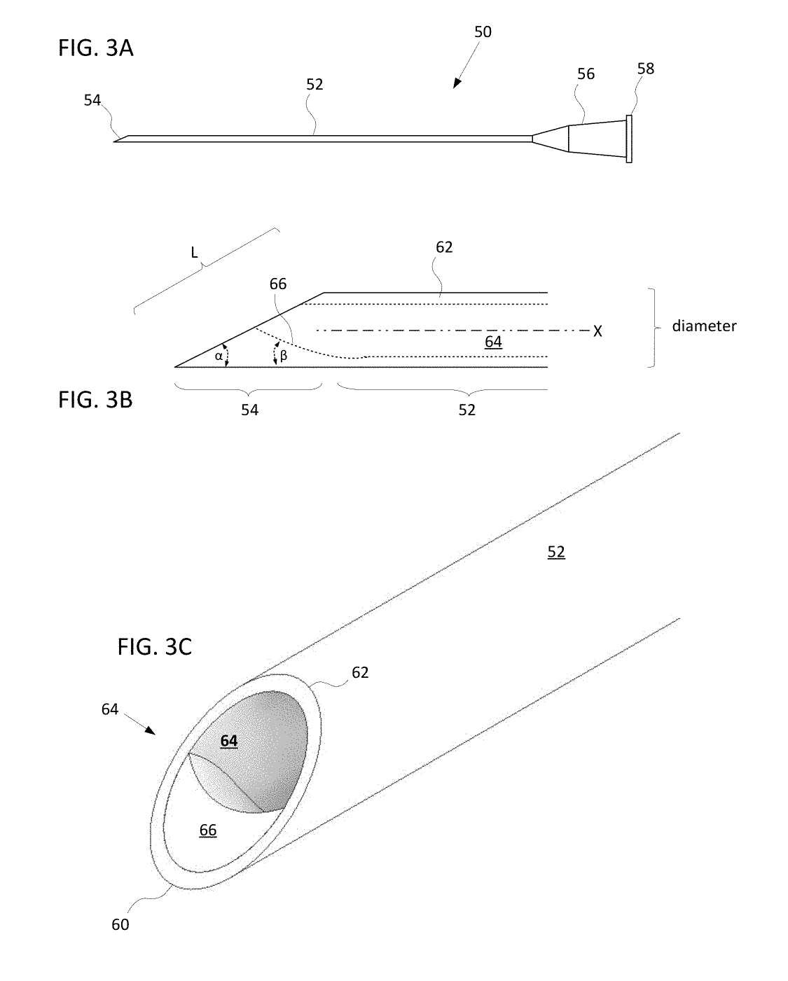

To assist in understanding the invention, reference is made to the accompanying drawings to shown by way of illustration specific embodiments in which the invention may be practiced. FIGS. 3A-3C show an example of my invention. FIG. 3A shows a side view of a needle 50 with a needle barrel that comprises a shaft portion 52. At its distal end, the needle barrel has a beveled tip 54. At the proximal end of the needle 50, there is a hub 56 having a Luer-type collar 58 for connecting with a Luer-type connection fitting.

FIG. 3B shows a close-up, see-through view of the distal portion of the needle 50. FIG. 3C shows a close-up, perspective view of the distal portion of the needle 50. Seen in these figures is the cylindrical wall 62 of the needle barrel and the central longitudinal axis X of the needle barrel. The wall 62 defines a hollow channel 64 of the needle barrel. At the bevel portion 54 of the needle barrel, there is a lancet tip 60 that provides a sharp edge for cutting into skin and body tissue. The bevel 54 may be cut at any suitable angle α to provide the lancet tip 20. In some embodiments, the bevel angle is in the range of 8-25°. Because it is cut at an angle, the bevel has a length (designated by “L”) longer than the diameter of the needle barrel. In some embodiments, the bevel length is 1.25-2 times the diameter of the needle barrel; in some cases, 1.5-1.75 times the diameter; in some cases, 1.5-5 times the diameter; in some cases, 2-5 times the diameter.

Unlike a conventional needle, at the bevel portion 54, there is an internal angled lip 66 that creates a ramp-like path for a guidewire exiting the opening at the bevel portion 54 of the needle 50. In this embodiment, the lip 66 has an angle β in the range of 5-20°, and in some cases in the range of 7-15° (e.g. about 10°) as it extends away from the needle wall 62. In this embodiment, the lip 66 angle β increases as the lip 66 approaches the bevel edge to more than 10°, e.g. increasing to 25-90°, and more preferably 35-60°. The lip 66 may extend across the needle diameter by about 50% or 30-70% and may have a concave surface which smoothly merges with the needle wall 62.

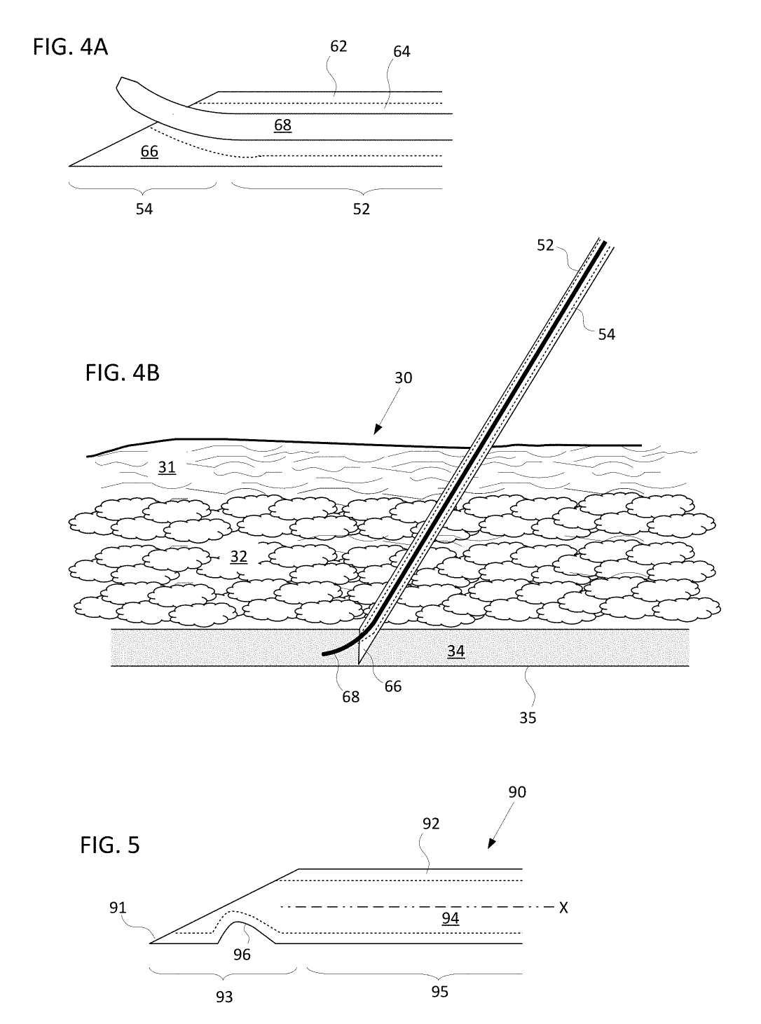

FIGS. 4A and 4B show how a guidewire 68 could be used with the needle 50. FIG. 4A shows a close-up, see-through view of the distal portion of the needle 50 with a guidewire 68 extending therethrough. As seen here, the channel 64 in the shaft portion 52 maintains a straight path, i.e. parallel to the central longitudinal axis of the needle barrel. Likewise, in the shaft portion 52 of the needle barrel, the guidewire 68 maintains a straight course, i.e. parallel to the central longitudinal axis of the needle barrel.

However, because of the lip 66, the channel 64 in the bevel portion 54 takes an oblique course relative to the central longitudinal axis of the needle barrel. Likewise, when exiting the needle barrel through the opening at the bevel portion 54, the lip 66 causes the guidewire 68 to bend and take an oblique course relative to the central longitudinal axis of the needle barrel.

FIG. 4B shows how the needle 50 could be used for providing the initial access path into the vein 35. As seen here, the needle 50 is punctured through the skin 30 at approximately 60° angle, advancing through the superficial fascia 31, through the fatty layer 32, and into the vein 35. To secure this access path into the vein 35, a blunt guidewire 68 is inserted through the needle hub and passed through the hollow needle shaft 52 until it exits from the bevel at the needle tip and into the lumen 34 of the vein 35. The angled lip 66 directs the guidewire 68 to exit the bevel at an oblique angle (relative to the central longitudinal axis of the needle barrel) into the lumen 34 of the vein 35. With the guidewire 68 held within the vein 35, the needle 50 is withdrawn backwards over the guidewire 68, leaving only the guidewire 68 in place. A central venous catheter can then be introduced into vein 35 over this guidewire 68.

As shown in FIG. 4B, by its design, the needle 50 can approach the targeted vein 35 at a steeper angle as compared to a conventional needle. This steeper approach angle shortens the distance of penetration through the skin 30 to reach the vein 35. This makes it easier for the operator to aim towards the target vein 35. This also makes it easier to locate the target vein 35 with ultrasound imaging and visualize placement of the needle barrel. In addition, shortening the distance that the needle 50 must traverse is safer because there is less possibility of inadvertently puncturing adjacent structures. Yet, even with this steeper approach, the guidewire 68 can exit from the needle shaft at an angle that allows entry into the 34 lumen of the vein 35.

In my invention, the internal lip could be formed by any suitable manufacturing technique, such as stamping, grinding, molding, cutting, or punching processes. The internal lip may be a unitary structure with the cylindrical wall or a separate structure from the cylindrical wall. FIG. 5 shows a particular example of how an internal lip could be formed. Shown here is a close-up, see-through view of the distal portion of a needle 90. A cylindrical wall 92 forms the barrel of the needle, which has a bevel portion 93 and a shaft portion 95. The barrel encloses a channel 94. On the face of the barrel with the lancet tip 91, a section of the cylindrical wall 92 is punched-in to create a dent. This dent results in a deflection in the cylindrical wall 92 that forms the internal lip 96 of the needle barrel.

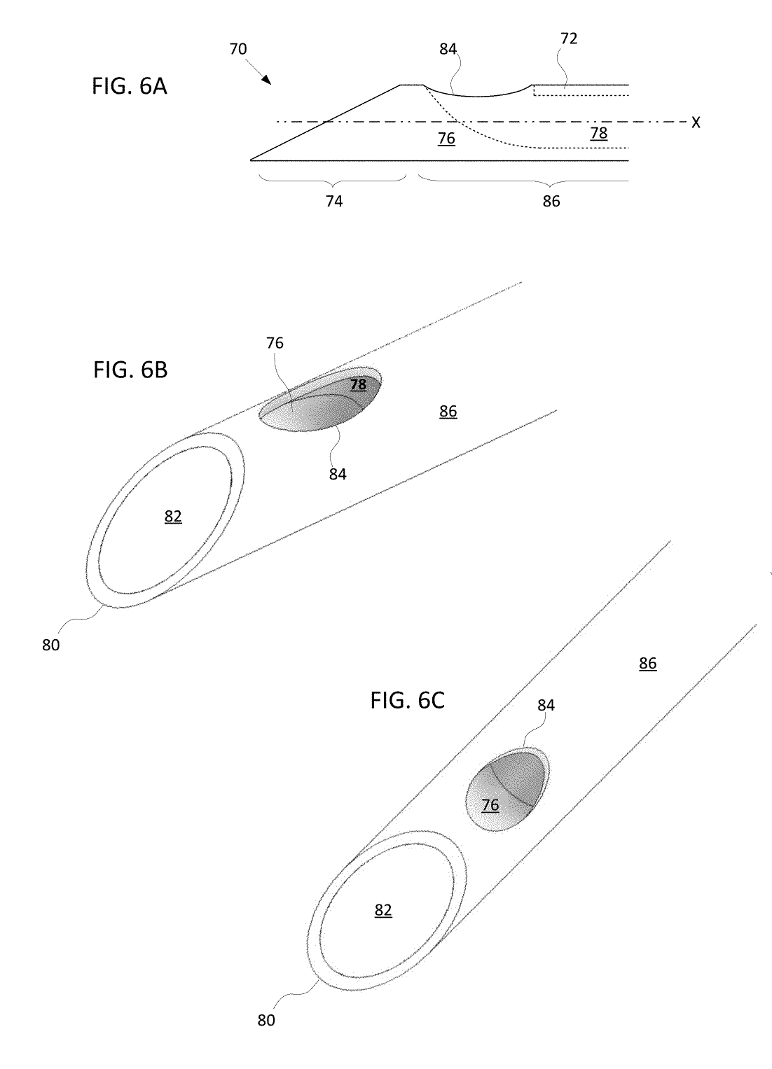

FIGS. 6A-6C show another example of my invention. FIG. 6A shows a close-up, see-through view of the distal portion of the needle 70. FIGS. 6B and 6C show close-up, perspective views of the distal portion of the needle 70. Seen in these figures is the cylindrical wall 72 of the needle barrel, which comprises a shaft portion 86 and a bevel portion 74. A central longitudinal axis X extends along the needle barrel. The wall 72 defines a hollow channel 78 of the needle barrel. At the bevel portion 74 of the needle barrel, there is a lancet tip 80 that provides a sharp edge for cutting into skin and body tissue. The bevel portion 74 may be cut at any suitable angle α to provide the lancet tip 80, such as the angles given for the embodiment represented in FIGS. 3A-3C. The bevel portion 74 may have any suitable length, such as the lengths given for the embodiment represented in FIGS. 3A-3C.

Unlike a conventional needle, on the shaft portion 86 of the needle barrel near the bevel portion 74, there is a side opening 84 made through the wall 72 of the needle shaft 86. Within the channel 78, there is an internal angled lip 76 that forms a ramp leading towards this side opening 84. This provides a path such that a guidewire advanced through the hollow channel 78 will exit out the side of the needle shaft 86. Because the guidewire exits out the side of the needle shaft 86, the bevel portion 74 has a solid face 82 (instead of being hollow).

The internal lip 76 may have any suitable angle β as it extends away from the needle wall 72, such as the angles given for the embodiment represented in FIGS. 3A-3C above. Optionally, again as in the embodiment represented in FIGS. 3A-3C, there can be an increase in the angle β as the lip 76 approaches the bevel edge. The lip 76 may extend across the needle diameter by about 50% or 30-70% and may have a concave surface which smoothly merges with the needle wall 72.

The foregoing description and examples have been set forth merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance.

Modifications of the disclosed embodiments incorporating the spirit and substance of the invention may occur to persons skilled in the art, and such modifications are within the scope of the invention. Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A hollow bore needle comprising:
a barrel defined by a cylindrical wall, the barrel having a distal end;
a channel defined by the barrel extending along a central longitudinal axis;
at the distal end of the barrel, a bevel portion of the barrel that comprises a lancet tip;
extending proximally from the bevel portion, a shaft portion of the barrel;
within the barrel at the bevel portion, an internal lip that defines a ramp having an incline relative to the central longitudinal axis or the cylindrical wall at the shaft portion of the barrel.

2. The hollow bore needle of claim 1, wherein the path of the channel at the shaft portion of the barrel is parallel to the central longitudinal axis and at the bevel portion, the path is oblique relative to the central longitudinal axis.

3. The hollow bore needle of claim 1, wherein the outer surface of the barrel is parallel and coaxial to the central longitudinal axis along the entire length of the barrel, including the bevel portion.

4. The hollow bore needle of claim 1, wherein the barrel has a diameter in the range of 18-24 gauge.

5. The hollow bore needle of claim 1, wherein barrel has a length in the range of 2.5-12 cm.

6. The hollow bore needle of claim 1, wherein the barrel is made of a material comprising stainless steel.

7. The hollow bore needle of claim 1, wherein the internal lip is angled in the range of 5-20° relative to the cylindrical wall or the central longitudinal axis.

8. The hollow bore needle of claim 1, wherein the internal lip is located within 2.5 cm of the distal end of the barrel.

9. The hollow bore needle of claim 1, further comprising a Luer-type hub at the proximal end.

10. The hollow bore needle of claim 1, wherein the bevel portion is angled in the range of 8-25° relative to the central longitudinal axis of the barrel.

11-14. (canceled)

15. A vascular access kit comprising:
(a) a hollow bore needle comprising:
a barrel defined by a cylindrical wall, the barrel having a distal end;
a channel defined by the barrel extending along a central longitudinal axis;
at the distal end of the barrel, a bevel portion of the barrel that comprises a lancet tip;
extending proximally from the bevel portion, a shaft portion of the barrel;
within the barrel at the bevel portion, an internal lip that defines a ramp having an incline relative to the central longitudinal axis or the cylindrical wall at the shaft portion of the barrel;
(b) a guidewire configured to travel through the channel of the hollow bore needle.

16. The vascular access kit of claim 15, further comprising a syringe.

17. The vascular access kit of claim 15, wherein the guidewire has a diameter in the range of 0.014 inches (0.36 mm)-0.050 inches (1.3 mm).

18. A method of providing an access path into a blood vessel of a patient, comprising:
(a) having a hollow bore needle comprising:
a barrel defined by a cylindrical wall, the barrel having a distal end;
a channel defined by the barrel extending along a central longitudinal axis;
at the distal end of the barrel, a bevel portion of the barrel that comprises a lancet tip;
extending proximally from the bevel portion, a shaft portion of the barrel;
within the barrel at the bevel portion, an internal lip that defines a ramp having an incline relative to the central longitudinal axis or the cylindrical wall at the shaft portion of the barrel;
(b) applying the needle to the patient's skin;
(c) puncturing the skin and advancing the needle until the bevel portion of the barrel enters the lumen of the blood vessel;
(d) advancing a guidewire through the channel of the needle and into the lumen of the blood vessel;
(e) withdrawing the needle over the guidewire, leaving the distal end of the guidewire in place within the lumen of the blood vessel.

19. The method of claim 18, wherein the needle is applied at an angle of greater than 45° relative to the skin surface or the blood vessel.

20. The method of claim 18, wherein the guidewire exits the barrel and into the lumen of the blood vessel at an oblique angle relative to the central longitudinal axis.
